(19) United States
(12) Patent Application Publication
(10) Pub. No.: US 2025/0366920 A1
(43) Pub. Date: Dec. 4, 2025

(54) LASER INTRAVASULAR LITHOTRIPSY DEVICE FOR STENOSIS CALCIFIED LESIONS

(71) Applicant: Shockwave Medical, Inc., Santa Clara, CA (US)

(72) Inventors: Steven Yihlih PENG, Fremont, CA (US); Thomas Charles HASENBERG, Campbell, CA (US)

(73) Assignee: Shockwave Medical, Inc., Santa Clara, CA (US)

(21) Appl. No.: 18/680,853

(22) Filed: May 31, 2024

(51) Int. Cl. A61B 18/26; A61B 17/00

(52) U.S. Cl. CPC A61B 18/26; A61B 2017/00154; A61B 2017/0019; A61B 2018/263

(57) ABSTRACT

Laser IVL systems and methods are disclosed. A laser IVL system can include at least one light energy source and a catheter. The catheter can include an elongate sheath, an enclosure sealed to a distal end of the elongate sheath, and at least one optical fiber contained within the elongate sheath. The enclosure may have a fill volume less than 10 mL and may be fillable with a fluid. The at least one optical fiber can be optically coupled to receive light from the at least one light energy source and configured to transmit the received light at a light emitting region of the at least one optical fiber into a distal region of the catheter that is enclosed by the enclosure to generate shock waves in the fluid.

### FIELD

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. The energy from this electrical discharge enters the surrounding fluid faster than the speed of sound, generating an acoustic shock wave. To describe this mechanism in greater detail, first, delivery of current to the electrodes generates one or more ionization bubbles on the electrode surface. Subsequently, current arcs across the ionization bubble or bubbles from one electrode to another electrode across the spark gap therebetween, resulting in the energy release and shock wave generation. In addition, the energy from the electrical discharge may create one or more rapidly expanding and collapsing cavitation bubbles. Collapse of the cavitation bubble(s) and associated colliding of liquid walls may generate additional acoustic waves and, potentially, one or more microjets. The acoustic energy propagates radially outward and modify calcified plaque within the blood vessels.

For laser generation of acoustic shock waves, a laser pulse is transmitted into and absorbed by a fluid within the catheter to generate a shock wave and/or cavitation bubbles. These events may occur when light from a laser light source is absorbed by the fluid surrounding the light emitting region of the optical fiber(s), generating a shock wave. Subsequently, heating and evaporation of water in the media may create a cavitation bubble. Collapse of the cavitation bubble and associated colliding of liquid walls may generate additional acoustic waves and, potentially, one or more microjets. The acoustic energy intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that generate shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with a fluid (using a relatively low pressure of 2-4 atm) so that the balloon expands to contact the lesion but is not an inflation pressure that substantively displaces the lesion. Energy can then be delivered to the catheter inside the balloon to produce acoustic shock waves that propagate through the walls of the angioplasty balloon and into the lesions, for example by applying voltage pulses across electrodes (if the energy source is electrohydraulic) or by transmitting a laser pulse into the fluid (if the energy source is a laser). Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure.

Many shock wave generating devices include shock wave emitters spaced along the length (e.g., along the longitudinal axis) of the device's body. These devices are useful for treating buildup of calcified plaque along the length of the inner wall of a body lumen such as a blood vessel. However, in many cases, calcified buildup in body structures (e.g., Mitral Annular Calcification (MAC) and Chronic Total Occlusions (CTOs) or fibrotic tissue buildup surrounding pacemaker leads in cardiac tissue) can become very thick, significantly narrowing the body lumen. While electrohydraulic shock wave generating devices that generate forward-directed shock waves and/or cavitation bubbles have been developed for the treatment of such calcifications and fibrotic tissues, the treatment capabilities of these devices can become inconsistent over time as the electrodes used to produce the shock waves degrade.

### SUMMARY

Provided are laser IVL systems that generate forward-directed shock waves and/or cavitation bubbles for targeting stenosis-causing calcified lesions. A disclosed laser IVL system can include a catheter that can include an elongate sheath that is capped at its distal end with an enclosure that is fillable with a fluid. Optical fiber(s) contained within the sheath can be optically coupled to receive light from a light energy source (e.g., a laser) and configured to transmit the received light into the distal region of the catheter that is enclosed by the conical enclosure. When the enclosure is filled with fluid, light energy transmitted into the enclosed distal region can generate shock waves and/or cavitation bubbles in the fluid. Energy from these shock waves and/or cavitation bubbles can be transmitted through the enclosure and into a lesion to treat stenosis.

The laser IVL systems described herein can repeatedly generate highly consistent shock waves with characteristics that are consistent and reproduceable over time. Properties of the light that is used to produce the shock waves (e.g., the power, the frequency, the wavelength, the pulse width, etc.) can be tuned to target specific types of lesions in order to optimize treatment efficiency and outcomes. Some embodiments of the provided laser IVL systems are equipped with multiple preprogrammed light settings that enable physicians to adapt the catheter to treat different types of lesions as needed. Other embodiments of the disclosed laser IVL systems include manual or automated controls for adjusting properties of the light as needed in real time.

A laser IVL system can comprise at least one light energy source and a catheter. The catheter can include an elongate sheath, an enclosure sealed to a distal end of the elongate sheath and having a fill volume less than 10 mL, and at least one optical fiber contained within the elongate sheath. The enclosure may be fillable with a fluid. The at least one optical fiber can be optically coupled to receive light from the at least one light energy source and configured to transmit the received light at a light emitting region of the at least one optical fiber into a distal region of the catheter that is enclosed by the enclosure to generate shock waves in the fluid.

In some embodiments, a distal end of the enclosure tapers in width. The enclosure can have a conical shape. A diameter of the base of the enclosure may be between 0.5 mm and 10 mm. A length of the enclosure may be between 1 mm and 10 mm.

A distance from the light emitting region of the at least one optical fiber to the enclosure can be at least 1.0 mm. In some embodiments, the light emitting region of the at least one optical fiber comprises a distal end of the at least one optical fiber. In other embodiments, the light emitting region of the at least one optical fiber comprises an evanescent portion of the at least one optical fiber.

The at least one optical fiber can be positioned proximally to an inner surface of the elongate sheath. In some embodiments, the catheter comprises at least two optical fibers. A spatial arrangement of the at least two optical fibers in the catheter can be radially symmetric. For example, the at least two optical fibers can be arranged in two or more concentric rings. Alternatively, a spatial arrangement of the at least two optical fibers in the catheter may not be radially symmetric.

In some embodiments, a first property of light coupled into a first optical fiber and a second optical fiber of the at least two optical fibers is controlled independently for the first optical fiber and the second optical fiber. The laser IVL system can comprise least two light energy sources; a first optical fiber of the at least two optical fibers can be optically coupled to receive light from a first light energy source of the at least two light energy sources and a second optical fiber of the at least two optical fibers can be optically coupled to receive light from a second light energy source of the at least two light energy sources.

The catheter can further comprise an elongate support member contained within the elongate sheath. An outer surface of the elongate support member may include one or more longitudinal channels. The at least one optical fiber may be disposed within a respective channel of the one or more longitudinal channels. The elongate support member may comprise an axial lumen for receiving a guide wire.

The laser IVL system can include a controller for controlling one or more properties of the light provided to the at least one optical fiber by the at least one light energy source. The light from the at least one light energy source may have a wavelength between 760 and 2200 nm. The at least one light energy source may provide pulses of light. A peak power of the pulses of light may be between 325 W and 375 W. A pulse width of each pulse of light may be between 10 ns and 500 us. The at least one light energy source may provide the pulses of light with a pulse repetition rate between 200 Hz and 1 kHz, for example a pulse repetition rate between 700 Hz and 800 Hz. The at least one light energy source may be a laser light source.

In some embodiments, for a wavelength of light provided by the at least one light energy source, the fluid has an absorption coefficient of at least 100 cm⁻¹. The catheter may include at least one fluid lumen fluidically coupled to receive the fluid from a fluid source.

A laser IVL method can comprise advancing a catheter into a bodily structure. The catheter may comprise an elongate sheath, a enclosure sealed to a distal end of the elongate sheath and having a fill volume less than 10 mL, and at least one optical fiber contained within the elongate sheath. The enclosure may be filled with a fluid. The at least one optical fiber may be optically coupled to receive light from at least one light energy source and configured to transmit the received light at a light emitting region of the optical fiber into a distal region of the catheter that is enclosed by the enclosure to generate shock waves in the fluid. The laser IVL method may further comprise positioning the enclosure of the catheter adjacent to an occlusion in the bodily structure and providing a plurality of light pulses from the at least one light energy source to the at least one optical fiber to generate one or more shock waves in the fluid. The occlusion may be a total chronic occlusion. The shock waves may propagate through the fluid and impinge on the occlusion. The catheter may then be advanced further into the bodily structure, and the enclosure may be repositioned adjacent to the occlusion. A second plurality of light pulses may be provided from the at least one light energy source to the at least one optical fiber to generate one or more additional shock waves in the fluid. The one or more additional shock waves propagate through the fluid and impinge on the occlusion.

In some embodiments, the method further comprises controlling one or more properties of the light provided by the at least one light energy source to the at least one optical fiber. The one or more properties of the light can be controlled based on one or more characteristics of the occlusion. If the catheter comprises at least two optical fibers, the one or more properties of the light that is provided to a first optical fiber of the at least two optical fibers may be controlled independently of the one or more properties of the light that is provided to a second optical fiber of the at least two optical fibers.

The method can include filling the enclosure with the fluid. The catheter may include at least one fluid lumen fluidically coupled to receive fluid from a fluid source. Filling the conical enclosure with the fluid may comprise providing a volume of fluid from the fluid source to the at least one fluid lumens.

A distal end of the enclosure may taper in width. In some embodiments, the enclosure has a conical shape. The at least one optical fiber can be positioned proximally to an inner surface of the elongate sheath. The light emitting region of the at least one optical fiber can comprise a distal end of the at least one optical fiber or an evanescent portion of the at least one optical fiber.

### BRIEF DESCRIPTION OF THE FIGURES

The invention will now be described, by way of example only, with reference to the accompanying drawings, in which:

FIG. 1 shows a schematic of a forward-firing laser IVL system, according to some embodiments.

FIG. 2A shows a semi-transparent perspective view of a forward-firing catheter for performing laser IVL, according to some embodiments.

FIG. 2B shows a semi-transparent perspective view of a forward-firing catheter for performing laser IVL, according to some embodiments.

FIG. 3 shows a cross-sectional front view of a forward-firing catheter for performing laser IVL, according to some embodiments.

FIG. 4 shows a cross-sectional front view of another forward-firing catheter for performing laser IVL, according to some embodiments.

FIG. 5 shows a method for treating a stenotic lumen with a forward-firing laser IVL system, according to some embodiments.

FIG. 6 shows a computer system, according to some embodiments.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Disclosed are catheters, systems, and methods for generating forward-biased or forward-directed shock waves and/or cavitation bubbles for targeting stenosis-causing calcified, fibrotic, or mixed morphology lesions. A catheter may include an elongate sheath that is capped at its distal end with an enclosure that is fillable with a fluid. Optical fiber(s) contained within sheath can be optically coupled to receive light from a light energy source (e.g., a laser) and configured to transmit the received light into the distal region of the catheter that is enclosed by the conical enclosure. When the enclosure is filled with fluid, light energy transmitted into the enclosed distal region can generate shock waves and/or cavitation bubbles in the fluid. Energy from these shock waves and/or cavitation bubbles can be transmitted through the enclosure and into a lesion to treat stenosis.

The catheters described herein can repeatedly generate highly consistent shock waves with characteristics that are consistent and reproduceable over time. Properties of the light that is used to produce the shock waves (e.g., the power, the frequency, the wavelength, the pulse width, etc.) can be tuned to target specific types of lesions in order to optimize treatment efficiency and outcomes. Some embodiments of the provided laser IVL systems are equipped with multiple preprogrammed light settings that enable physicians to adapt the catheter to treat different types of lesions as needed. Other embodiments of the disclosed laser IVL systems include manual or automated controls for adjusting properties of the light as needed in real time.

Any of the systems, methods, techniques, and/or features disclosed herein may be combined, in whole or in part, with any other systems, methods, techniques, and/or features disclosed herein.

Reference to “about” or “approximately” a value or parameter herein includes (and describes) variations of that value or parameter per se. For example, description referring to “approximately X” or “about X” includes description of “X” as well as variations of “X”.

When a range of values or values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

“Bodily structures” can include any portion of any body part, for example any portion of a circulatory system, a urinary tract, or a digestive system.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, in U.S. Publication No. 2021/0085383, and in U.S. patent application Ser. No. 18/586,299, all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward firing catheter designs can be found in U.S. Pat. Nos. 10,966,737, 11,478,261, and 11,596,423, in U.S. Publication Nos. 2023/0107690 and 2023/0165598, and in U.S. patent application Ser. No. 18/524,575, all of which are incorporated herein by reference in their entireties.

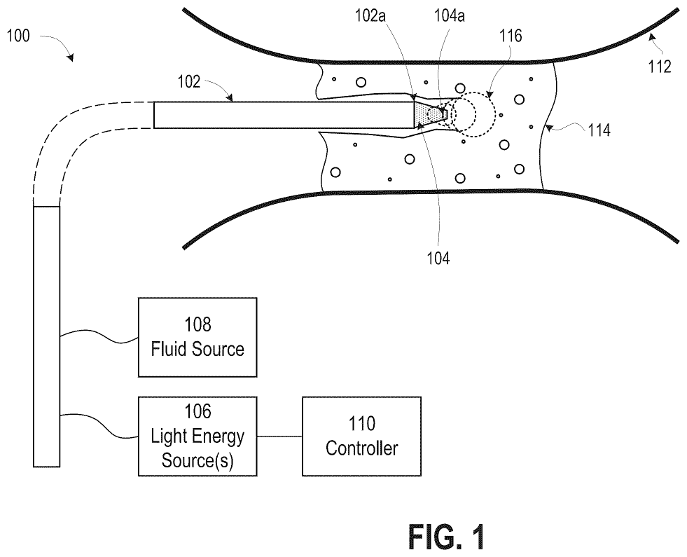

FIG. 1 depicts an exemplary laser IVL system 100. As shown, system 100 can include a catheter 102 that can generate forward-directed shock waves (i.e., shock waves and/or cavitation bubbles directed distally of catheter 102) for targeting and breaking up an occlusion 114 in a body lumen or body structure 112. Catheter 102 generates shock waves by transmitting light energy received from a light energy source 106 into fluid contained in an enclosure 104 that encloses a distal region of catheter 102.

Body lumen or body structure 112 may be any portion of any body part. In some embodiments, body lumen or body structure 112 is a portion of a circulatory system. In other embodiments, body lumen or body structure 112 is a portion of a digestive system. Occlusion 114 can be any occlusion that is treatable using lithotripsy, for example a kidney stone, a gallstone, a bladder stone, a ureter stone, a urethral stone, a bezoar, or a calcified lesion in a blood vessel. In some embodiments, occlusion 114 may partially, mostly, or entirely block body lumen or body structure 112, or may constrict or narrow body lumen or body structure 112 such that catheter 102 cannot advance through body lumen or body structure 112 without first breaking down portions of occlusion 114. Catheter 102 can be positioned in body lumen or body structure 112 using a guide wire.

Enclosure 104 may be sealed to a distal end 102a of catheter 102 and may be configured to be both fillable with fluid and transmissible to shock waves 116 generated when light energy from light energy source 106 is transmitted into the fluid. The fluid with which enclosure 104 is filled may be a conductive fluid such as saline that is provided by a fluid source 108. When enclosure 104 is filled with fluid and pressurized, enclosure 104 may maintain a substantively constant volume and profile. In some embodiments, enclosure 104 may be filled with up to 10 milliliters (mL) of fluid. In some embodiments, enclosure 104 may be filled with up to 6 mL of fluid. The fluid may allow shock waves 116 to propagate distally from a distal end 102a of catheter 102, through the outer surface of enclosure 104, and into a target lesion (e.g., occlusion 114).

Enclosure 104 may have a tapered geometry to benefit trackability. In some embodiments, a distal end 104a of enclosure 104 tapers in width or diameter. In some embodiments, enclosure 104 has a conical or approximately conical shape. In some embodiments, enclosure 104 has a frustoconical shape. In some embodiments, enclosure 104 has a bullnose shape.

Light can be transmitted from light energy source 106 and into enclosure 104 through one or more optical fibers contained in catheter 102. Light energy source 106 can include a laser, for example a InGaP diode laser, a Tm:YAG laser, a InGaAs diode laser, a Nd:YAG laser, a pulsed dye laser, a holmium YAG laser, or a thulium fiber laser, or any other suitable laser. The light transmitted into the optical fibers of catheter 102 from light energy source 106 may have a near-infrared wavelength, that is, a wavelength between about 760 nm and about 2200 nm. For example, the light transmitted into the optical fibers of catheter 102 from light energy source 106 may have a wavelength of about 800 nm, 820 nm, 840 nm, 860 nm, 880 nm, 900 nm, 920 nm, 940 nm, 960 nm, 980 nm, 1000 nm, 1020 nm, 1040 nm, 1060 nm, 1080 nm, 1100 nm, 1120 nm, 1140 nm, 1160 nm, 1180 nm, 1200 nm, 1220 nm, 1240 nm, 1260 nm, 1280 nm, 1300 nm, 1320nm, 1340 nm, 1360 nm, 1380 nm, 1400 nm, 1420 nm, 1440 nm, 1460 nm, 1480 nm, or 2200 nm. Advantageously, near-infrared light exhibits strong absorption in water so may be suitable for use with aqueous solutions such as saline for geniting shock waves.

In some embodiments, wavelengths of light other than near-infrared wavelengths may be chosen. For example, an ultraviolet light energy source may be selected in combination with a contrast media.

Light energy source 106 may be configured to provide pulses of light. The width of each light pulse may be less than the time required for a bubble to reach equilibrium in the fluid contained in enclosure 104 if the fluid was free (e.g., not enclosed). In some embodiments, the width of each pulse is between 1 ns and 30 ns, between 5 ns and 25 ns, between 10 ns and 25 ns, or between 15 ns and 20 ns, for example about 16 ns, about 17 ns, about 18 ns, about 19 ns, or about 20 ns. In other embodiments, the width of each pulse is between 5 ns and 500 μs, for example 100 ns, 500 ns, 1 μs, 50 μs, 75 μs, 100 μs, 150 μs, 200 μs, 250 μs, 300 μs, 350 μs, 400 μs, or 450 μs. The peak power of each light pulse can be between 100 W and 500 W, for example approximately 150 W, 200 W, 250 W, 300 W, 350 W, 400 W, or 450 W. The pulse repetition rate may be between about 1 Hz and 1 kHz, for example about 150 Hz, 200 Hz, 250 Hz, 300 Hz, 350 Hz, 400 Hz, 450 Hz, 500 Hz, 550 Hz, 600 Hz, 650 Hz, 700 Hz, 710 Hz, 720 Hz, 730 Hz, 740 Hz, 750 Hz, 760 Hz, 770 Hz, 780 Hz, 790 Hz, 800 Hz, 810 Hz, 820 Hz, 830 Hz, 840 Hz, 850 Hz, 900 Hz, or 950 Hz.

Light energy source 106 can coupled to a controller 110 that is configured to control one or more properties of the light received by catheter 102. Controller 110 may be operable by a user (e.g., a physician) and may include a computer system. Controller 110 can allow the user to (continuously or step-wise) adjust a wavelength, a pulse width, a peak pulse power, and/or a pulse repetition rate of the light. In some embodiments, one or more of the light properties are independently adjustable.

In some embodiments, controller 110 provides the user with one or more pre-set, selectable light settings, each of which indicates a particular set of light properties. In some embodiments, before beginning a treatment session, the type of tissue that is to be treated can be determined, e.g., using autofluorescence (AF) or diffuse reflective spectroscopy (DRS) techniques. During the treatment session (e.g., prior to inserting catheter 102 into body lumen or body structure 112), the user can select a light setting using controller 110 to choose the light properties that are most appropriate for the lesion that is being targeted. Controller 110 may comprise a user interface (e.g., a graphical user interface) configured to display one or more recommended light settings (e.g., in a menu) based on the determined lesion type. This may allow system 100 to be easily adapted to treat lesions of different shapes, sizes, and densities.

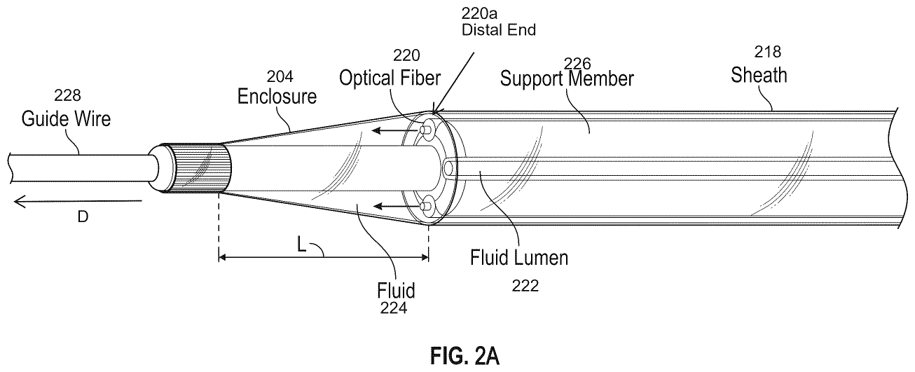

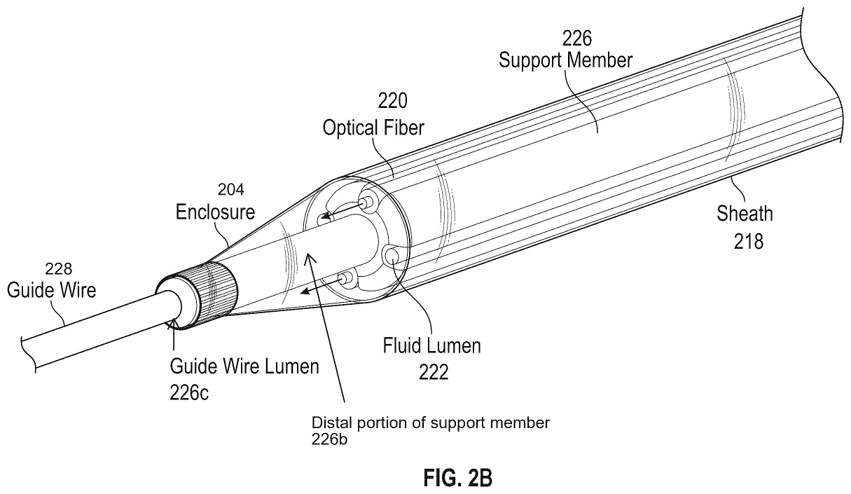

Semi-transparent views of an exemplary catheter 202 are provided in FIGS. 2A-2B. Catheter 202 can be a component of a laser IVL system such as system 100 shown in FIG. 1. As shown, catheter 202 can include an elongate sheath 218 that is sealed at a distal end 204a by a enclosure 204 that is fillable with a fluid 224. Contained in elongate sheath 218 may be one or more optical fibers 220, each of which may be optically coupled to receive light from a light energy source (e.g., light energy source 106 shown in FIG. 1). Optical fiber(s) 220 may terminate at distal end 220a such that light propagating through each optical fiber is transmitted into a distal region of catheter 202 that is enclosed by enclosure 204. When enclosure 204 is filled with fluid 224 and light is transmitted into the enclosed distal region, shock waves may be produced in fluid 224. Energy from the shock waves may be directed through the outer surface of enclosure 204 in a distal direction (indicated by arrow “D” in FIGS. 2A-2B) and into the region surrounding catheter 202. If catheter 202 is positioned adjacent to a lesion such that distal end 202a is facing the lesion, the energy from the shock waves can be transmitted into the lesion, facilitating the lesion's breakdown.

Enclosure 204 can be formed from a flexible or a rigid membrane, for example a polymer membrane. As shown, enclosure 204 can have a tapered geometry, for example a conical or frustoconical geometry. The conical or frustoconical shape of enclosure 204 may direct energy from shock waves generated in fluid 224 in the distal and radial directions. Enclosure 204 may be sealed to distal end 204a of elongate sheath 218 at its base, which may have a circular or elliptical shape that matches the cross-sectional shape of elongate sheath 218. A length L of enclosure 204 can be between about 1 mm and about 10 mm, for example about 1.25 mm, 1.5 mm. 1.75 mm, 2 mm, 2.25 mm, 2.5 mm, 2.75 mm, 3 mm, 3.25 mm, 3.5 mm, 3.75 mm, 4 mm, 4.25 mm, 4.5 mm, 4.75 mm, 5 mm, 5.25 mm, 5.5 mm, 5.75 mm, 6 mm, 6.25 mm, 6.5 mm, 6.75 mm, 8 mm, 8.25 mm, 8.5 mm, 8.75 mm, 9 mm, 9.25 mm, 9.5 mm, or 9.75 mm. In some embodiments, a length L of enclosure 204 is less than 1 mm or greater than 10 mm.

The wall of enclosure 204 may comprise a non-porous material configured to facilitate the efficient transfer of energy from the shock waves to treatment sites. In some embodiments, the wall of enclosure 204 is formed entirely from a non-porous material. In other embodiments, the wall of enclosure 204 is formed predominantly (but not entirely) from a non-porous material. For example, the majority of the wall of enclosure 204 may be formed from a non-porous material and a minority portion of the wall of enclosure 204 may be formed from a material. In other embodiments, the wall of enclosure 204 is partially formed from a non-porous porous material. For instance, half of the surface area of the wall of enclosure 204 may be formed from a non-porous material and half of the surface area of the wall of enclosure 204 may be formed from a porous material. Enclosure 204 can have an acoustic impedance that is (approximately) matched with acoustic properties of fluid 224 in order to efficiently transmit shock waves through the wall of enclosure 204 to the target tissue.

According to aspects of the disclosure, the absorption depth and absorption coefficient of the light in the fluid are factors in determining a minimum spacing of light emitting regions of one or more optical fibers from the enclosure. In other words, absorption depth of the light may be determined by light wavelength and the choice of media, and the minimum spacing the light emitting regions from the enclosure may be at least the absorption depth. In some embodiments, the minimum spacing is 0.5 mm. In some embodiments, the minimum spacing is 1.0 mm.

In some embodiments, in contrast to lithotripsy systems having emitters exposed to the environment, enclosure 204 mitigates thermal injury to soft tissue and reduces cavitation stresses by limiting expansion of the vapor bubbles produced during shock wave generation to the interior of enclosure 204. Vapor bubbles may hit the enclosure wall before reaching their maximum potential size, inducing collapse and, as a result, reducing cavitation stress and preventing soft tissue injury that can be caused by tensile stresses during cavitation bubble collapse.

Optical fiber(s) 220 may be positioned proximally to an inner surface of elongate sheath 218. In some embodiments, optical fiber(s) 220 are supported by an elongate support member 226 contained within elongate sheath 218. Each optical fiber may be disposed within a longitudinal channel 226a in an outer surface of elongate support member 226. Support member 226 can include an axial lumen 226c along its longitudinal axis for receiving a guide wire 228. The diameter of support member 226 may decrease in a distal portion 226b that passes through enclosure 204 to maximize the volume of fluid 224 that can be contained in enclosure 204 and to minimize interference of support member 226 with the shock waves while still enabling support member 226 to support to guide wire 228. Optical fiber(s) 220 may be positioned using shrink tubing and/or adhesive.

Fluid 224 can be provided to enclosure 204 by one or more fluid lumens 222. Each fluid lumen 222 may be fluidically coupled to receive fluid from a fluid source (e.g., fluid source 108 shown in FIG. 1). Like optical fiber(s) 220, fluid lumen(s) 222 may be positioned proximally to an inner surface of elongate sheath 218. If catheter 202 includes elongate support member 226, each fluid lumen 222 may be disposed in a longitudinal channel in an outer surface of support member 226.

Fluid 224 may have a high absorption coefficient for the wavelength of light that is provided to optical fiber(s) 330 by the light energy source. For example, the absorption coefficient of fluid 224 for light with a wavelength of approximately 2 μm (e.g., 1.99, 2.01, 2.02, 2.03, 2.04, or 2.05 μm) may be approximately 100 cm⁻¹ (e.g., 99.9, 99.99, 100.01, 100.02, or 100.03 cm⁻¹). In some embodiments, the absorption coefficient of fluid 224 for a given wavelength of light provided to optical fiber(s) 330 is at least 1 cm⁻. In some embodiments, for a higher absorption coefficient of fluid 224, a distance between a light emitting region of optical fiber(s) 330 and the enclosure may be shorter.

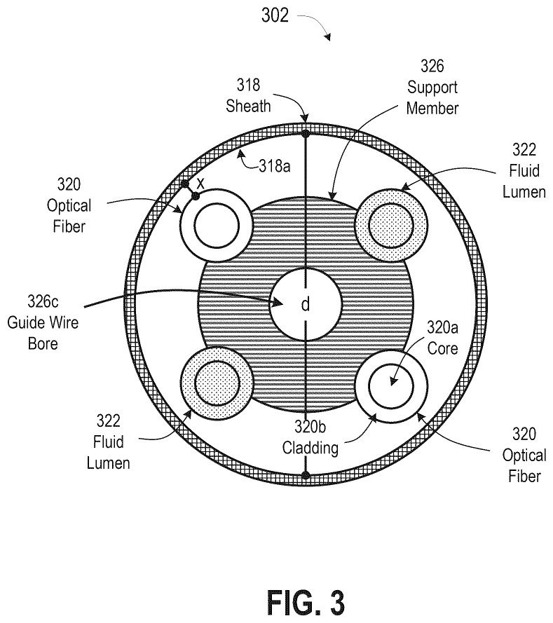

FIG. 3 shows a cross-sectional front view of an exemplary catheter 302. Like catheter 202 shown in FIGS. 2A-2B, catheter 302 comprises a sheath 318. Sheath 318 may have a circular cross-section. The diameter d of sheath 318 can be between about 1 mm and about 5 mm, for example about 1.25 mm, 1.5 mm, 1.75 mm, 2 mm, 2.25 mm, 2.5 mm, 2.75 mm, 3 mm, 3.25 mm, 3.5 mm, 3.75 mm, 4 m, 4.25 mm, 4.5 mm, or 4.75 mm. In some embodiments the diameter d of sheath 318 is less than 1 mm or greater than 5 mm. The cross-sectional shape and size of sheath 318 may match the cross-sectional shape and size of the base of the enclosure that encloses the distal region of the catheter (e.g., enclosure 204 shown in FIGS. 2A-2B).

Sheath 318 may contain one or more optical fibers 320. Each optical fiber 320 may comprise a core 320a encased in a cladding 320b. A light energy source (e.g., light energy source 106 shown in FIG. 1) may be optically coupled to provide light to each optical fiber 320 at an angle relative to the longitudinal axis of the optical fiber such that, when the light hits the interface between core 320a and cladding 320b, the light undergoes total internal reflection within core 320a and is propagated along the length of the optical fiber.

Core 302a and cladding 302b may comprise the same materials or may have different material compositions. In some embodiments, both core 302a and cladding 302b comprise silica glass.

The index of refraction of cladding 302b may be greater than 2, between 1 and 2,between 1 and 1.9, between 1 and 1.8, between 1 and 1.7, between 1 and 1.6, or between 1 and 1.5. For example, the index of refraction of cladding 302b may be approximately 1.4, 1.41, 1.42, 1.43, or 1.44. In some embodiments, the index of refraction of cladding 302b is between 1.4 and 1.42, for example approximately 1.41, 1.411, 1.412, 1.413, 1.414, 1.415, 1.416, 1.417, 1.418, or 1.419. In some embodiments, the index of refraction of cladding 302b is between 1.419 and 1.42, for example approximately 1.4191, 1.4192, 1.4193, 1.4194, 1.4195, 1.4196, 1.4197, 1.4198, or 1.4199. Cladding 302b may have a lower index of refraction (for a given wavelength of light) than core 302a.

The index of refraction of core 302a may be greater than 2, between 1 and 2, between 1 and 1.9, between 1 and 1.8, between 1 and 1.7, between 1 and 1.6, or between 1 and 1.5. For example, the index of refraction of core 302a may be approximately 1.4, 1.41, 1.42, 1.43, or 1.44. In some embodiments, the index of refraction of core 302a is between 1.43 and 1.44, for example approximately 1.431, 1.432, 1.433, 1.434, 1.435, 1.436, 1.437, 1.438, or 1.439. In some embodiments, the index of refraction for core 302a is between 1.436 and 1.437, for example approximately 1.4361, 1.4362, 1.4363, 1.4364, 1.4365. 1.4367, 1.4368, or 1.4369. Core 302a may have a higher index of refraction (for a given wavelength of light) than cladding 302b. In some embodiments, a difference between the index of refraction of core 302a and cladding 302b for a given wavelength is about 0.01, 0.011, 0.012, 0.013, 0.014, 0.015, 0.016, 0.017, 0.018, 0.019, or 0.02.

Each optical fiber 320 may be disposed in a longitudinal channel in an outer surface of a support member 326. In some embodiments, each optical fiber is positioned proximal to an inner surface 318a of sheath 318. In some embodiments, a separation distance x between each optical fiber and inner surface 318a of sheath 318 is less than or equal to 1 mm, 0.75 mm, 0.5 mm, 0.25 mm, 0.1 mm, 0.05 mm, or 0.025 mm. In some embodiments, each optical fiber 320 is in contact with an inner surface 318a of sheath 318.

One or more fluid lumens 322 may be configured to be fluidically coupled to receive fluid from a fluid source. Fluid lumen(s) 322 can be positioned proximal to inner surface 318a of sheath 318 and may be disposed in longitudinal channel(s) in an outer surface of support member 326. In some embodiments, an inner diameter of each fluid lumen is between 0.1 and 0.3 mm, for example about 0.15, 0.16, 0.17, 0.18, 0.19, 0.2, 0.21, 0.22, 0.23, 0.24, or 0.25 mm.

A catheter may include any number of optical fibers. In some embodiments, a catheter includes only a single optical fiber. In other embodiments, a catheter includes multiple optical fibers. For example, a catheter can include at least two, at least 3, at least 4, at least 5, at least 6, at least 7, at least 8, at least 9, or at least 10 optical fibers.

In catheters that include multiple optical fibers, the transmission of light through each optical fiber, as well as the properties of said light, can be controlled independently. For example, if a catheter includes two optical fibers, light can be transmitted alternately through the first optical fiber and the second optical fiber, at different pulse rates or patterns through the first optical fiber and the second optical fiber, at different power levels, at different wavelengths, at different polarizations, and/or at different optical modes as coupled into the optical fibers. In some embodiments, optical fibers may be controlled individually and/or in one or more groups.

In catheters that include multiple optical fibers, the optical fibers can be optically coupled to receive light from the same light energy source. The optical fibers can be connected to the light energy source through one or more optical switches. Closing a switch between a particular optical fiber and the light energy source may allow light to be transmitted from the light energy source and into that optical fiber. Likewise, opening a switch between an optical fiber and the light energy source may prevent light from being transmitted from the light energy source and into that optical fiber. In other embodiments, an aperture, filter wheel, or other blocking/filtering device may be selectively positioned between the one or more optical fibers and the light energy source. One or more portions of the filtering device may be transparent to light from the light energy source and one or more portions of the filtering device may block light from the light energy source. To transmit light through a particular optical fiber, a portion of the filtering device that transmits light from the light energy source may be positioned in an optical path between the optical fiber and the light energy source. To block transmission of light through a particular optical fiber, a portion of the filtering device that blocks light from the light energy source may be positioned in an optical path between the optical fiber and the light energy source.

In some embodiments, a laser IVL system includes multiple light energy sources. Each light energy source may be optically coupled to provide light to a particular optical fiber or set of optical fibers in the catheter. The light energy sources can be independently controlled, either by a single controller or by separate controllers.

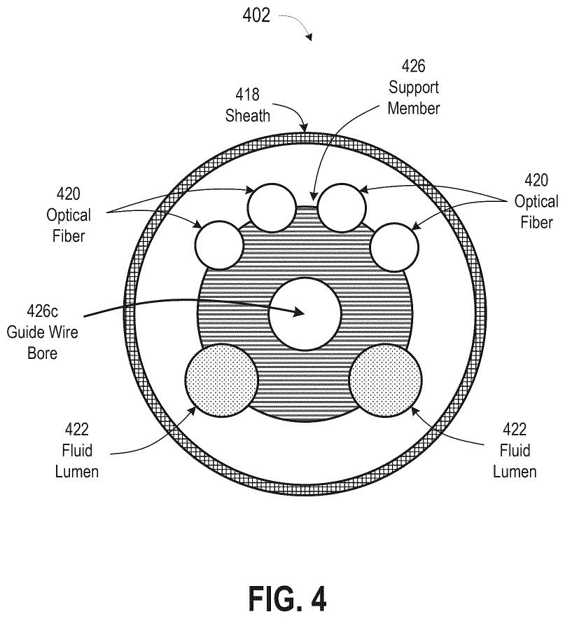

In a catheter that include multiple optical fibers, the fibers may be positioned parallel to the longitudinal axis of the catheter. The fibers can be spatially distributed within the catheter in numerous ways. For example, the optical fibers can be arranged in a radially symmetric pattern about the longitudinal axis of the catheter so that the catheter can emit shock waves symmetrically about its longitudinal axis. In some embodiments, the optical fibers are arranged in one or more concentric rings centered on the longitudinal axis of the catheter. Alternatively, the optical fibers can be arranged in a pattern that is not radially symmetric. For example, the optical fibers can be clustered in a single sector of the catheter, e.g., as depicted in FIG. 4, which shows a catheter 402 that includes four optical fibers 420 arranged in an arc in one half region of catheter 402 so that catheter 402 emits shock waves asymmetrically about its longitudinal axis. In some embodiments, optical fibers are spatially distributed within a catheter so that the shock waves generated using each optical fiber constructively interfere with the shock waves generated using the other optical fibers.

Each optical fiber in the catheter can comprise a light emitting region through which light that is coupled into the optical fiber is transmitted out of the optical fiber and into the fluid contained in the enclosed distal end of the catheter. In some embodiments, the light emitting region of an optical fiber is a distal end of the optical fiber.

In other embodiments, the light emitting region of an optical fiber comprises an evanescent region of the fiber. The evanescent region may be a region of the fiber that is configured to allow a portion (e.g., at least 10%, at least 15%, at least 30%, at least 40%, or at least 50% of the optical power) of the light propagating through the core to leak out of the optical fiber into the fluid. The portion of the light that leaks out of the optical fiber in the evanescent region may be the evanescent field that penetrates into the cladding due to the total internal reflection of the light in the fiber core. The fiber cladding in the evanescent region may be thinner than the fiber cladding in non-evanescent regions of the fiber to enable the evanescent field to be transmitted out of the cladding and into the fluid.

In some embodiments, a (shortest) distance between a light emitting region of an optical fiber and the enclosure of the catheter is at least 1.0 mm. In some embodiments, a (shortest) distance between a distance between a light emitting region of an optical fiber and the enclosure of the catheter is about 1.5 mm, 2 mm, 2.5 mm, 3 mm, 3.5 mm, or 4 mm. In some embodiments, a distance between a light emitting region of an optical fiber and the enclosure of the catheter is at least 4 mm.

In some embodiments, in a catheter that includes multiple optical fibers, light can be transmitted into a subset of the optical fibers. For example, to target an irregularly shaped lesion, light may be transmitted into a subset of the optical fibers that are positioned in a particular region of the catheter (e.g., a particular sector or “pie slice” of the catheter).

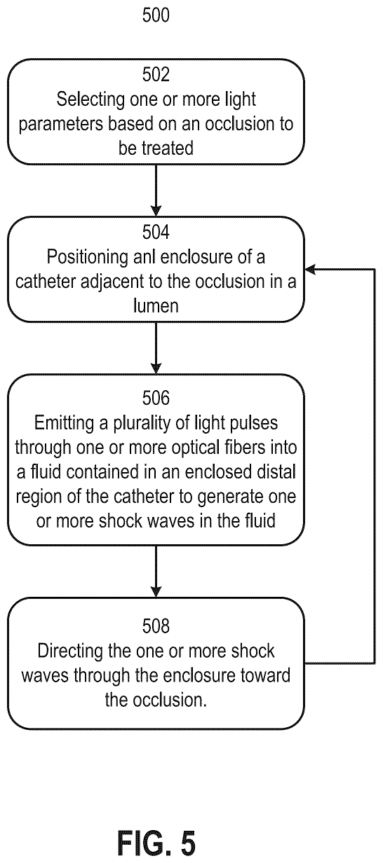

A method 500 for using a laser IVL system such as system 100 (see FIG. 1) comprising a catheter such as catheter 202 (see FIGS. 2A-2B), catheter 302 (see FIG. 3), or catheter 402 (see FIG. 4) is provided in FIG. 5. As shown, at the start of a treatment session, one or more light parameters for light to be provided to the optical fiber(s) in the catheter may be selected (step 502 of method 500). A physician may select the appropriate light parameters using a controller for the light energy source (e.g., controller 110 for light energy source 106 shown in FIG. 1), for example by individually selecting values for the light parameters or by selecting a setting option from a set of available setting options. The selection may be made based on a number of factors, for instance based on the type or characteristics of the lesion that is being targeted, based on the location of the occlusion, or based on the patient's health data (e.g., the patient's age, health conditions, etc.). In some embodiments, the controller is configured to automatically recommend one or more light parameter settings by analyzing one or more of the aforementioned factors (e.g., by leveraging a machine learning model).

After the light parameters have been selected, the catheter may be inserted into the bodily structure or lumen that contains the lesion, and a distal portion of the catheter may be positioned adjacent to the occlusion (step 504 of method 500). Specifically, the fluid-containing distal region of the catheter that is enclosed by the enclosure (e.g., enclosure 204 shown in FIG. 2) may be positioned adjacent to the occlusion such that the tip of the enclosure faces the occlusion. A plurality of light pulses may then be emitted through the optical fiber(s) in the catheter and into the fluid contained in the enclosed distal region of the catheter to generate one or more shock waves in the fluid (step 506 of method 500). The light pulses may have properties that match the light parameters selected in step 502. The generated shock waves may be directed through the enclosure and toward the occlusion (step 508 of method 500). As the occlusion breaks apart, the catheter may be advanced further into the bodily structure or lumen, repositioned adjacent to the occlusion, and caused to emit additional shock waves until a sufficient amount of the occlusion has been removed.

In some embodiments, a catheter for treating lesions in body lumen includes an enclosure and shock wave emitters within the enclosure where one or more of the shock wave emitters generate shock waves optically and one or more shock wave emitters generate shock waves electrohydraulically. The enclosed emitters may include one or more light emitting regions of one or more optical fibers optically connected to a laser light source (as described above) and one or more electrode pairs electrically connected to a high voltage source. In some embodiments, the light emitting regions are more distally positioned within the enclosure than the electrode pairs. In some embodiments, the light emitting regions are distal ends of the optical fibers that emit light distally and the electrode pairs are circumferentially positioned about a central axis of the catheter and emit shock waves predominantly radially away from the central axis. In some embodiments, the electrohydraulic shock wave emitters are forward firing. In some embodiments, the electrohydraulic shock wave emitters are side firing. Other examples of electrohydraulic shock wave emitters (e.g., emitters that generate shock waves at electrode pairs) are described in U.S. Pub. No. 2021/0085347, which is incorporated herein by reference. In various embodiments, a computer system can be used in combination with the

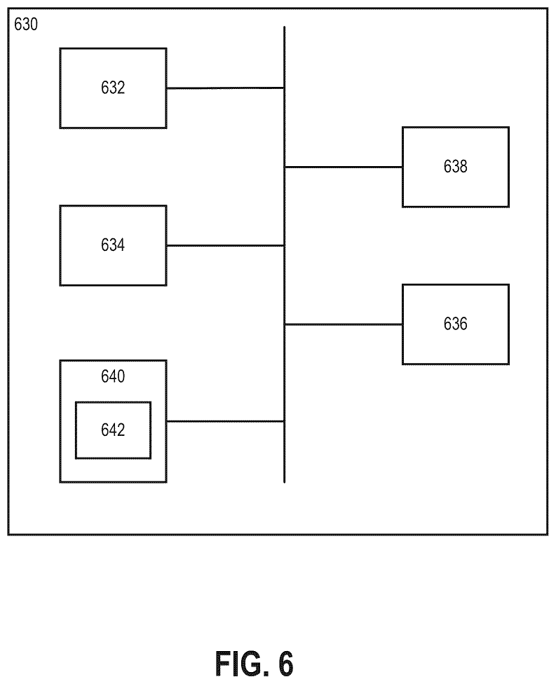

catheter devices described herein, for example to tune or otherwise control laser parameters (e.g., laser frequency, pulse frequency, intensity, etc.), to receive and process data associated with the catheter devices or a lesion for which a catheter device is being used (e.g., imaging sensor data, pressure sensor data, etc.), to interface with one or more control devices and/or graphical user interfaces usable to control the system(s) described herein, and/or to interface with one or more network/connected devices to send and/or receive data (e.g., control data, data generated by monitoring system usage, sensor data, etc.). For example, a computer system may be a component of a controller (e.g., controller 110 shown in FIG. 1) for controlling a light energy source to which the optical fibers in a catheter are coupled. An exemplary computer system 630 is provided in FIG. 6. System 630 can be any suitable type of processor-based system, such as a personal computer, workstation, server, handheld computing device (portable electronic device) such as a phone or tablet, or dedicated device. The system 630 can include, for example, one or more of input device 634, output device 636, one or more processors 632, storage 640, and communication device 638. Input device 634 and output device 636 can generally correspond to those described above and can either be connectable or integrated with the computer.

Input device 634 can be any suitable device that provides input, such as a push-button switch, a touch screen, keyboard or keypad, mouse, gesture recognition component of a virtual/augmented reality system, or voice-recognition device. Output device 636 can be or include any suitable device that provides output, such as a display, touch screen, haptics device, virtual/augmented reality display, or speaker.

Storage 640 can be any suitable device that provides storage, such as an electrical, magnetic, or optical memory including a RAM, cache, hard drive, removable storage disk, or other non-transitory computer readable medium. Communication device 638 can include any suitable device capable of transmitting and receiving signals over a network, such as a network interface chip or device. The components of the computing system 630 can be connected in any suitable manner, such as via a physical bus or wirelessly.

Processor(s) 632 can be any suitable processor or combination of processors, including any of, or any combination of, a central processing unit (CPU), graphics processing unit (GPU), field programmable gate array (FPGA), programmable system on chip (PSOC), and application-specific integrated circuit (ASIC). Software 642, which can be stored in storage 640 and executed by one or more processors 632, can include, for example, the programming that embodies the functionality or portions of the functionality of the present disclosure (e.g., as embodied in the devices as described above)

Software 642 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 642 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 630 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 630 can implement any operating system suitable for operating on the network. Software 642 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The catheter devices described herein can be used for a variety of occlusions, including coronary occlusions, such as lesions in vasculature, or occlusions in the peripheral vasculature (e.g., above-the-knee, below-the-knee, iliac, carotid, etc.). For further examples, similar designs may be used for treating soft tissues, such as cancer and tumors (i.e., non-thermal ablation methods), blood clots, fibroids, cysts, organs, scar and fibrotic tissue removal, or other tissue destruction and removal. Catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

The elements and features of the exemplary catheters discussed above may be rearranged, recombined, and modified, without departing from the present invention. Furthermore, numerical designators such as “first”, “second”, “third”, “fourth”, etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It should be noted that the elements and features of the example catheters illustrated throughout this specification and drawings may be rearranged, recombined, and modified without departing from the present invention. For instance, the present disclosure is intended to include catheters having a variety of balloon configurations. The number, placement, and spacing of the evanescent regions can be modified without departing from the subject invention. Further, the number, placement, and spacing of balloons of catheters can be modified without departing from the subject invention.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A laser intravascular lithotripsy (IVL) system comprising:
at least one light energy source;
a catheter comprising:
an elongate sheath;
a frustoconical enclosure comprising a proximal base having a first diameter and a distal end having a second diameter, wherein the first diameter is larger than the second diameter, wherein the proximal base of the frustoconical enclosure is sealed to a distal end of the elongate sheath, the rigid frustoconical enclosure being fillable with a fluid; and
at least one optical fiber contained within the elongate sheath, the at least one optical fiber optically coupled to receive light from the at least one light energy source and configured to transmit the received light at a light emitting region of the at least one optical fiber into a distal region of the catheter that is enclosed by the frustoconical enclosure to generate shock waves in the fluid, wherein the shock waves are directed through an outer surface of the frustoconical enclosure and are forward-biased in a distal direction; and
a controller configured to control at least a wavelength, pulse width, peak pulse power, or pulse repetition rate of the light transmitted to the distal region of the catheter that is enclosed by the frustoconical enclosure.

2. (canceled)

3. (canceled)

4. The laser IVL system of claim 1, wherein the first diameter of the proximal base of the rigid-frustoconical enclosure is between 0.5 mm and 10 mm.

5. The laser IVL system of claim 1, wherein a length of the rigid frustoconical enclosure is between 1 mm and 10 mm.

6. The laser IVL system of claim 1, wherein a distance from the light emitting region of the at least one optical fiber to the rigid frustoconical enclosure is at least 1.0 mm.

7. The laser IVL system of claim 1, wherein the light emitting region of the at least one optical fiber comprises a distal end of the at least one optical fiber.

8. (canceled)

9. The laser IVL system of claim 1, wherein the at least one optical fiber is positioned proximally to an inner surface of the elongate sheath.

10. The laser IVL system of claim 1, wherein the catheter comprises at least two optical fibers.

11. The laser IVL system of claim 10, wherein a spatial arrangement of the at least two optical fibers in the catheter is radially symmetric.

12. The laser IVL system of claim 11, wherein the at least two optical fibers are arranged in two or more concentric rings.

13. The laser IVL system of claim 10, wherein a spatial arrangement of the at least two optical fibers in the catheter is not radially symmetric.

14. The laser IVL system of claim 10, further comprising a controller configured to control a first property for light coupled into a first optical fiber and to control the first property for light coupled into a second optical fiber of the at least two optical fibers, such that the first property for the first optical fiber and the first property for the second fiber are independently adjusted with respect to one another.

15. The laser IVL system of claim 10, comprising at least two light energy sources, wherein a first optical fiber of the at least two optical fibers is optically coupled to receive light from a first light energy source of the at least two light energy sources, and wherein a second optical fiber of the at least two optical fibers is optically coupled to receive light from a second light energy source of the at least two light energy sources.

16. The laser IVL system of claim 1, wherein the catheter further comprises:
an elongate support member contained within the elongate sheath, wherein an outer surface of the elongate support member comprises one or more longitudinal channels, wherein the at least one optical fiber is disposed within a respective channel of the one or more longitudinal channels.

17. The laser IVL system of claim 16, wherein the elongate support member comprises an axial lumen for receiving a guide wire.

18. (canceled)

19. The laser IVL system of claim 1, wherein the light from the at least one light energy source has a wavelength between 760 and 2200 nm.

20. The laser IVL system of claim 1, wherein the at least one light energy source provides pulses of light.

21. The laser IVL system of claim 20, wherein a peak power of the pulses of light is between 325 W and 375 W.

22. The laser IVL system of claim 20, wherein a pulse width of each pulse of light is between 10 ns and 500 μs.

23. The laser IVL system of claim 20, wherein the at least one light energy source provides the pulses of light with a pulse repetition rate between 200 Hz and 1 kHz.

24. The laser IVL system of claim 23, wherein the pulse repetition rate is between 700 Hz and 800 Hz.

25. The laser IVL system of claim 1, wherein the at least one light energy source is a laser light source.

26. The laser IVL system of claim 1, wherein the frustoconical enclosure is filled with the fluid, and, for a wavelength of light provided by the at least one light energy source, the fluid has an absorption coefficient of at least 100 cm⁻¹.

27. (canceled)

28. A laser intravascular lithotripsy (IVL) method comprising:
advancing a catheter into a bodily structure, the catheter comprising:
an elongate sheath,
a rigid frustoconical enclosure comprising a proximal base having a first diameter and a distal end having a second diameter, wherein the first diameter is larger than the second diameter, wherein the proximal base of the rigid frustoconical enclosure is sealed to a distal end of the elongate sheath, wherein the rigid frustoconical enclosure is filled with a fluid, and
at least one optical fiber contained within the elongate sheath, the at least one optical fiber optically coupled to receive light from at least one light energy source and configured to transmit the received light at a light emitting region of the optical fiber into a distal region of the catheter that is enclosed by the rigid frustoconical enclosure to generate shock waves in the fluid, wherein the shock waves are directed through an outer surface of the rigid frustoconical enclosure and are forward-biased in a distal direction;
positioning the rigid frustoconical enclosure of the catheter adjacent to an occlusion in the bodily structure; and
providing a plurality of light pulses from the at least one light energy source to the at least one optical fiber to generate one or more shock waves in the fluid, wherein the shock waves propagate through the fluid and impinge on the occlusion.

29. The method of claim 28, further comprising controlling one or more properties of the light provided by the at least one light energy source to the at least one optical fiber.

30. The method of claim 29, wherein the one or more properties of the light are controlled based on one or more characteristics of the occlusion.

31. The method of claim 29, wherein the catheter comprises at least two optical fibers, and wherein the one or more properties of the light that is provided to a first optical fiber of the at least two optical fibers are controlled by a controller independently of the one or more properties of the light that is provided to a second optical fiber of the at least two optical fibers.

32. The method of claim 28, further comprising:
advancing the catheter further into the bodily structure;
repositioning the rigid frustoconical enclosure of the catheter adjacent to the occlusion;
providing a second plurality of light pulses from the at least one light energy source to the at least one optical fiber to generate one or more additional shock waves in the fluid, wherein the one or more additional shock waves propagate through the fluid and impinge on the occlusion.

33. The method of claim 28, further comprising filling the rigid frustoconical enclosure with the fluid.

34. The method of claim 33, wherein the catheter further comprises at least one fluid lumen fluidically coupled to receive fluid from a fluid source, and wherein filling the rigid frustoconical enclosure with the fluid comprises providing a volume of fluid from the fluid source to the at least one fluid lumens.

35. The method of claim 28, wherein a distal end of the frustoconical enclosure tapers in width.

36. (canceled)

37. The method of claim 28, wherein the at least one optical fiber is positioned proximally to an inner surface of the elongate sheath.

38. The method of claim 28, wherein the light emitting region of the at least one optical fiber comprises a distal end of the at least one optical fiber.

39. The method of claim 28, wherein the light emitting region of the at least one optical fiber comprises an evanescent portion of the at least one optical fiber.

40. The method of claim 28, wherein the occlusion is a total chronic occlusion.
